(19) United States
(12) Patent Application Publication
(10) Pub. No.: US 2019/0388681 A1
(43) Pub. Date: Dec. 26, 2019

(54) INFORMATION PROCESSING DEVICE, ELECTRICAL TREATMENT DEVICE, SYSTEM, AND PROGRAM

(71) Applicant: OMRON HEALTHCARE Co., Ltd., Muko-shi (JP)

(72) Inventors: Mitsuru SAMEJIMA, Kyoto (JP); Yui WATANABE, Kyoto (JP); Makoto TABATA, Kyoto (JP); Tetsuya SATO, Kyoto (JP); Shozo TAKAMATSU, Kyoto (JP)

(73) Assignee: OMRON HEALTHCARE Co., Ltd., Muko-shi (JP)

(21) Appl. No.: 16/563,297

(22) Filed: Sep. 6, 2019

Related U.S. Application Data
(63) Continuation of application No. PCT/JP2018/008754, filed on Mar. 7, 2018.

(30) Foreign Application Priority Data
Mar. 14, 2017 (JP) 2017-048520

(51) Int. Cl. A61N 1/36; A61N 1/372; A61N 1/04

(52) U.S. Cl. CPC A61N 1/3603; A61N 1/3606; A61N 1/046; A61N 1/37247

(57) ABSTRACT

An information processing device that determines a treatment content to be performed from a behavior tendency of a user itself of an electrical treatment device. The information processing device includes a control unit configured to control the information processing device, wherein the control unit includes a storage processing unit configured to acquire behavior information indicating a content of a behavior of a user of an electrical treatment device and store the behavior information in a storage unit, a tendency determination unit configured to determine a behavior tendency on the basis of the behavior information indicating the content of the behavior of the user stored in the storage unit, and a program determination unit configured to determine a treatment program for causing the electrical treatment device to perform a treatment content corresponding to the behavior tendency.

### TECHNICAL FIELD

The present disclosure relates to an information processing device, an electrical treatment device, a system, and a program, and particularly relates to an information processing device, an electrical treatment device, a system, and a program for treatment of the body.

### BACKGROUND ART

Known electrical treatment devices are configured to apply electrical stimulation to an area of the user's body by outputting a low-frequency pulse to the area via an electrode pad. For example, in a physical conditioning system of Patent Document 1 (JP 2009-539489 T), a treatment according to a programmed schedule that depends on an activated state of a sensed exercise period, patient rest or sleep, low heart rate level, something of the same kind and the like is applied.

In a living body stimulation device of Patent Document 2 (JP 2011-110117 A), treatment program information (connection/parameter information of an electrode block) is associated with a “patient ID”, a “medical professional ID”, “treatment history information”, and the like for treatment.

In a treatment device of Patent Document 3 (JP 2005-500109 T), a physician selects a desired parameter in a graphical user interface (GUI) window for selecting amplitude, frequency, waveform, treatment duration, and the like, and the treatment device is programmed to accurately process the parameters.

### CITATION LIST

### Patent Literature

Patent Document 1: JP 2009-539489 T

Patent Document 2: JP 2011-110117 A

Patent Document 3: JP 2005-500109 T

### SUMMARY OF INVENTION

### Technical Problem

The user of the electrical treatment device has a desire to receive electrical treatment based on tendencies of the user's body movement (such as whether they stand for a long time, walk often, and other tendencies) or tendencies of the user's operation of the electrical treatment device (such as which treatment modes are often used) in order to achieve reliable treatment results. However, in Patent Documents 1 to 3, a program for electrical treatment can be selected for each patient (or user), but no selection is performed on the basis of the tendencies of body movement of the user itself or the tendencies of operation by the user of the electrical treatment device.

In light of the circumstances described above, an object of an aspect of the present disclosure is to provide an information processing device, an electrical treatment device, a system, and a program that determine a treatment content to be performed from a behavior tendency of a user itself of the electrical treatment device.

### Solution to Problem

The information processing device according to this disclosure includes a control unit configured to control the information processing device, wherein the control unit includes a storage processing unit configured to acquire behavior information indicating a content of a behavior of a user of an electrical treatment device and store the behavior information in a storage unit, a tendency determination unit configured to determine a behavior tendency on the basis of the behavior information indicating the content of the behavior of the user stored in the storage unit, and a program determination unit configured to determine a treatment program for causing the electrical treatment device to perform a treatment content corresponding to the behavior tendency.

Preferably, the electrical treatment device includes a current generation unit configured to generate a current to be output to an area of a body according to the treatment program via an electrode portion configured to come into contact with the area, and the treatment program comprises a parameter for specifying a current waveform.

Preferably, the behavior includes an action of the user, and the behavior information includes a type of the action of the user and an amount of time or the number of times the action is performed.

Preferably, the behavior includes a user operation to the electrical treatment device, and the behavior information comprises a type of treatment mode to be specified by the user operation and an amount of time or the number of times the treatment mode has been specified.

An electrical treatment device according to another aspect of this disclosure includes a control unit configured to control the electrical treatment device, wherein the control unit is configured to acquire information indicating a behavior tendency of a user of the electrical treatment device and determine a treatment program for causing the electrical treatment device to perform a treatment content corresponding to a tendency indicated by the information acquired.

Preferably, the electrical treatment device comprises a current generation unit configured to generate a current to be output to an area of a body according to the treatment program via an electrode portion configured to come into contact with the area, and the treatment program comprises a parameter for specifying a current waveform.

A system according to another aspect of this disclosure includes

an information processing device; and

an electrical treatment device that communicates with the information processing device, wherein

the information processing device comprises a control unit configured to control the information processing device, and the control unit includes a tendency determination unit configured to determine a behavior tendency on the basis of behavior information indicating a content of a behavior of a user stored in a storage unit, a program determination unit configured to determine a treatment program for causing the electrical treatment device to perform a treatment content corresponding to the behavior tendency, and a transmission unit configured to transmit information of the treatment program determined to the electrical treatment device.

Preferably, the electrical treatment device comprises a current generation unit configured to generate a current to be output to an area of a body according to the treatment program via an electrode portion configured to come into contact with the area, and the treatment program comprises a parameter for specifying a current waveform.

According to another aspect of the disclosure, a program for causing a computer to execute an information processing method is provided. The information processing method includes acquiring behavior information indicating a content of a behavior of a user of an electrical treatment device and storing the behavior information in a storage unit, determining a behavior tendency on the basis of the behavior information stored in the storage unit, and determining a treatment program for causing the electrical treatment device to perform a treatment content corresponding to the behavior tendency.

Preferably, the electrical treatment device includes a current generation unit configured to generate a current to be output to an area of a body according to the treatment program via an electrode portion configured to come into contact with the area, and the treatment program comprises a parameter for specifying a current waveform.

### Advantageous Effects of Invention

According to this disclosure, a treatment content to be performed can be determined from a behavior tendency of a user itself of an electrical treatment device.

### BRIEF DESCRIPTION OF DRAWINGS

FIG. 1 is a diagram illustrating a schematic configuration of an information processing system 1 according to a first embodiment.

FIG. 2 is a diagram illustrating the appearance of a low-frequency treatment device 20 according to the first embodiment in association with a terminal device 10.

FIG. 3 is a diagram schematically illustrating an internal configuration of the low-frequency treatment device 20 according to the first embodiment.

FIG. 4 is a configuration diagram of a mobile device 21 according to the first embodiment.

FIG. 5 is a configuration diagram of a terminal device 10 according to the first embodiment.

FIG. 6 is a configuration diagram of a server 30 according to the first embodiment.

FIG. 7 is a diagram illustrating a functional configuration of the server 30 according to the first embodiment.

FIG. 8 is a diagram illustrating an example of the contents of a table 32 according to the first embodiment.

FIG. 9 is a diagram illustrating an example of the contents of a behavior information storage unit 31 according to the first embodiment.

FIG. 10 is a flowchart of processing according to the first embodiment.

### DESCRIPTION OF EMBODIMENTS

Embodiments will be described below with reference to the drawings. The same reference numerals are assigned to the same components and corresponding components, and redundant descriptions may not be repeated.

The terms used in a first embodiment are defined as follows.

“Content of behavior” includes types of user actions and types of user operations of the electrical treatment device.

“Behavior information” includes types of user actions and the amount of time or number of times the action is performed. Alternatively, types of user operations and the amount of time or the number of times the user operation is performed are included.

“Electrical treatment device” is a device that applies electrical stimulation to an area of the body via an electrode by applying a voltage to the electrode attached to the area, but the type of electrical treatment device is not limited thereto.

“Treatment program” is a program for causing an electrical treatment device to perform a treatment content, and includes a program to be executed by a processor of the electrical treatment device.

“Parameter of treatment program” is a value for specifying the treatment content to be performed by the treatment program and indicates, for example, a variable (value) provided as an argument to the treatment program. Accordingly, the contents of the treatment program can be modified by changing the parameter.

“Type of parameter” includes a waveform parameter, a voltage parameter, and a time parameter that specify the waveform of the current flowing through the electrode to the area of the body. Waveform parameters include parameters such as amplitude, pulse width, cycle, and duty ratio of the current waveform. Voltage parameters include a voltage value applied to the electrode. Time parameters include the treatment time (such as the amount of time the current flows through the electrode to the area). Note that the type of parameter is not limited to these.

### First Embodiment

### System Configuration

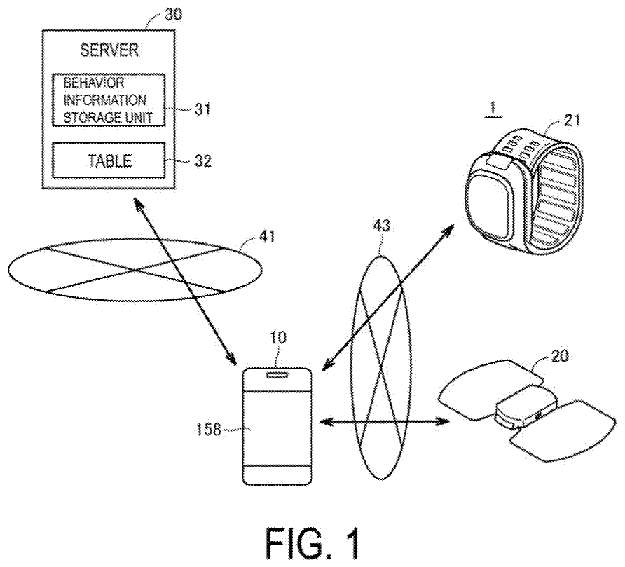

FIG. 1 is a diagram illustrating a schematic configuration of an information processing system 1 according to the first embodiment.

With reference to FIG. 1, the information processing system 1 includes a terminal device 10 used by a subject (also referred to as the user); a low-frequency treatment device 20, which is an example of an electrical treatment device; a watch-type mobile device 21, a server 30, and networks 41 and 43. The mobile device 21 includes a function of measuring biological information of the subject (e.g., blood pressure, number of steps, amount of activity, etc.).

The terminal device 10 is an example of an information processing device. The terminal device 10 is, for example, a smart phone including a touch panel. Note that the terminal device 10 may be another type of terminal device such as a folding type mobile telephone, a tablet terminal device, a personal computer (PC), a personal data assistant (PDA), and the like.

The network 41 includes various networks such as the Internet and a mobile terminal communication network to allow the terminal device 10 and the server 30 to connect to each other. The network 43 employs a short-range wireless communication system, typically Bluetooth (trade name) low energy (BLE), to connect the terminal device 10, the low-frequency treatment device 20, and the mobile device 21. However, the network 43 is not limited thereto, and a wired communication system may be employed, or other wireless communication systems such as a wireless local area network (LAN) may be employed.

The server 30 includes a behavior information storage unit 31 for accumulating and storing user behavior information and a table 32 in which parameters of the treatment program are registered.

### Configuration of Low-frequency Treatment Device 20

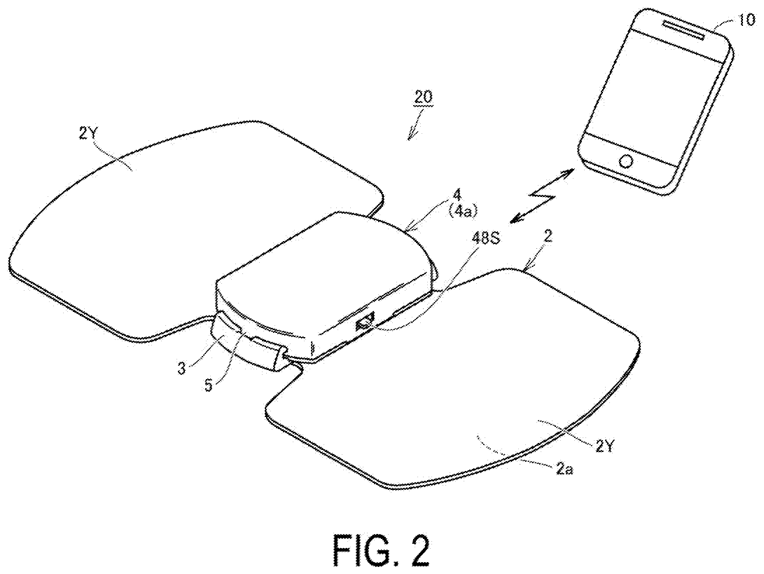

FIG. 2 is a diagram illustrating the appearance of the low-frequency treatment device 20 according to the first embodiment in association with the terminal device 10. Referring to FIG. 2, the low-frequency treatment device 20 includes a pad 2, a holder 3, and a main body portion 4. The low-frequency treatment device 20 is a so-called cordless type and is controlled according to control information received from the terminal device 10.

### Pad 2

The pad 2 is an example of an electrode portion that is configured to come into contact with an area of the body. The pad 2 has a sheet-like shape and is attached to an area of the user's body, specifically to the area to be treated or the like. A conductive layer 2a is provided on a surface (lower surface) of the outer surface of the pad 2 facing the body. The pad 2 is attached onto the user's skin using a conductive gel or the like, and a pulse current at a frequency corresponding to the treatment program is supplied to the user through the conductive layer 2a.

The pad 2 includes an attachment portion (not illustrated) and a treatment portion 2Y. The attachment portion is held by the holder 3. The holder 3 is positioned and disposed in the attachment portion. The treatment portion 2Y is provided on both the left and right sides of the attachment portion, and the conductive layer 2a is exposed on the surface of the treatment portion 2Y that faces the body. The conductive layer 2a is also exposed on the surface facing the main body portion 4, and the exposed portion constitutes an electrode.

### Main Body Portion 4

As illustrated in FIG. 2, the main body portion 4 includes a case 4a with a substantially rectangular parallelepiped shape as an outer cover. An engagement portion 5 is formed between the case 4a and the holder 3, and the main body portion 4 (case 4a) is detachably attached to the holder 3 by the engagement portion 5. The main body portion 4 is provided with a switch 48S that is operated by a user to control the low-frequency treatment device 20. The main body portion 4 also includes a display unit (not illustrated) for outputting information such as an operating state of the low-frequency treatment device 20.

With the main body portion 4 attached to the holder 3, the main body portion 4 supplies a low-frequency pulse current to the conductive layer 2a of the pad 2. Specifically, the main body portion 4 has a substrate, electric circuit mounted on the substrate, and the like built-in.

### Circuit Configuration of Low-frequency Treatment Device 20

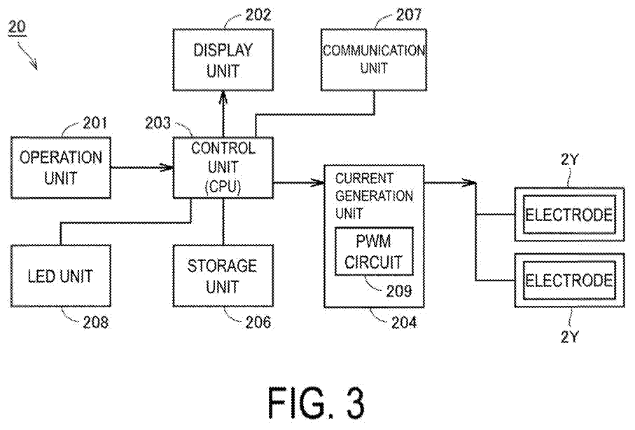

FIG. 3 is a diagram schematically illustrating an internal configuration of the low-frequency treatment device 20 according to the first embodiment. With reference to FIG. 3, the low-frequency treatment device 20 includes, as primary constituent elements, an operation unit 201, a display unit 202, a control unit 203 including a central processing unit (CPU), a current generation unit 204 including a pulse width modulation (PWM) circuit 209, a storage unit 206, a communication unit 207, and a light emitting diode (LED) unit 208. Although not illustrated, the low-frequency treatment device 20 includes a power source unit for supplying power to each constituent element. An alkaline battery, for example, is used as the power source unit. The power source unit stabilizes the battery voltage and generates a drive voltage to be supplied to each component.

The control unit 203 typically includes a central processing unit (CPU) or a micro-processing unit (MPU). The control unit 203 functions as a control unit that controls the operation of each component of the low-frequency treatment device 20 by reading out and executing the program stored in the storage unit 206.

The storage unit 206 is realized by random access memory (RAM), read-only memory (ROM), flash memory, and the like. The storage unit 206 stores programs (including a treatment program) to be executed by the control unit 203, data to be used by the control unit 203, and the like.

The operation unit 201 includes, for example, the switch 48S and various switches to receive a user's operation input to the low-frequency treatment device 20. When the user operates the operation unit 201, the control unit 203 receives the operation content and controls each component according to the received operation content.

The communication unit 207 includes a circuit for communicating with the terminal device 10. The communication unit 207 receives control information from the terminal device 10 and outputs the control information to the control unit 203. The control information includes the above-described parameters and an instruction command for setting the parameters in the treatment program.

In accordance with the command of the control information, the control unit 203 sets the parameters of the control information in the treatment program. Accordingly, the original parameters of the treatment program may be changed to change the treatment content to be performed by the treatment program. By executing the treatment program, the control unit 203 generates a PWM control signal (such as a voltage signal) according to the parameter after the change and outputs the generated signal to the PWM circuit 209.

The current generation unit 204 is a circuit that generates a current (hereinafter, also referred to as a treatment current) that flows from the pad 2 to the area of the user's body (specifically, the area to be treated). The PWM circuit 209 of the current generation unit 204 outputs a pulse current having an amplitude, a duty ratio (cycle), and the like indicated by the PWM control signal (i.e., the parameters), while keeping the pulse current constant and adjusting the amperage of the output current in accordance with the PWM control signal from the control unit 203. In this way, the waveform of the treatment current output from the current generation unit 204 (hereinafter, also referred to as a treatment waveform) is determined by the PWM control signal according to the parameter from the control unit 203. Note that the treatment current is not limited to a pulse current, and an alternating current may be used, or both types of current may be selectively used.

The treatment current output by the current generation unit 204 includes, for example, a pulse current with a low frequency. The pulse width of the treatment waveform with a low frequency pulse current is, for example, about 100 μsec for a small pulse width. Also, the maximum pulse amplitude is approximately 100 V, for example. The current generation unit 204 can change the pulse waveform (amplitude, pulse width, cycle, and the like) according to the treatment program to provide to the area various types of stimulation, such as “massage”, “tap”, “press”, and “rub”.

In addition, by changing the parameters of the treatment program, the treatment content for the area can be changed (adjusted). In particular, changing the pulse amplitude or pulse width allows the adjustment of the “intensity” (electrical stimulation intensity) of the stimulation such as “massage”, “tap”, “press”, “rub”, and the like, and changing the cycle of occurrence of a pulse group allows the adjustment of the “speed” of the stimulation such as “massage”, “tap”, “press”, “rub”, and the like.

Note that, in the first embodiment, a substantially constant electrical stimulation intensity is caused to be sensed regardless of the state of the body or the state of the area with which the electrode comes into contact (dry state, wet state, or the like). Thus, the low-frequency treatment device 20 may measure bioelectrical impedance and adjust the values of the parameters described above from the measured bioelectrical impedance. Note that a known method of measuring bioelectrical impedance may be used, and thus the description thereof is not repeated here.

### Configuration of Mobile Device 21

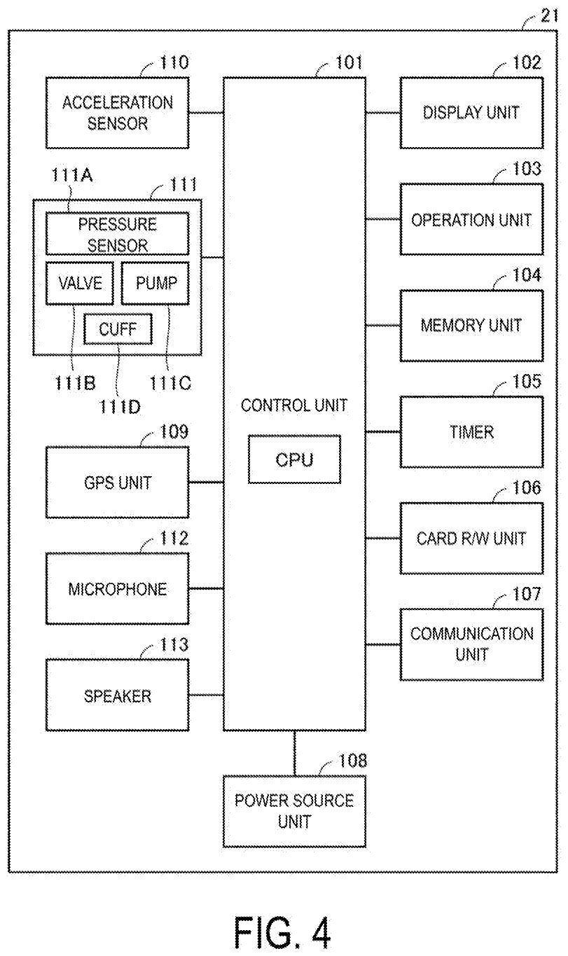

FIG. 4 is a configuration diagram of the mobile device 21 according to the first embodiment. Referring to FIG. 4, the mobile device 21 includes a control unit 101 including a central processing unit (CPU), a display unit 102 including a display such as a liquid crystal display, an operation unit 103 including a key button for receiving a user operation to the mobile device 21, a memory unit 104, a timer 105, a read/write (R/W) unit 106 that reads and writes information from/to a recording medium such as a memory card, a communication unit 107 for communicating with an external information processing device including the terminal device 10, a power source unit 108, a global positioning system (GPS) unit 109, an acceleration sensor 110, a blood pressure measurement unit 111, a microphone 112, and a speaker 113.

The memory unit 104 is realized by random access memory (RAM), read-only memory (ROM), flash memory, and the like. Programs executed by the control unit 101, data used by the control unit 101, and the like are stored in the storage region of the memory unit 104.

The acceleration sensor 110 measures the acceleration of the mobile device 21 and outputs the measured acceleration component to the control unit 101. The acceleration component includes, for example, voltage output proportional to acceleration in each direction of the X, Y, and Z axes.

The blood pressure measurement unit 111 includes a pressure sensor 111A, a valve 111B, a pump 111C, and a cuff 111D configured to be attached to the measurement area (arm). When measuring blood pressure, the control unit 101 controls the blood pressure measurement unit 111 and calculates a blood pressure value (for example, an indicator of systolic blood pressure SYS and diastolic blood pressure DIA) based on the cuff pressure output from the blood pressure measurement unit 111. Note that the pulse rate may also be calculated together with the blood pressure value.

### Configuration of Terminal Device 10

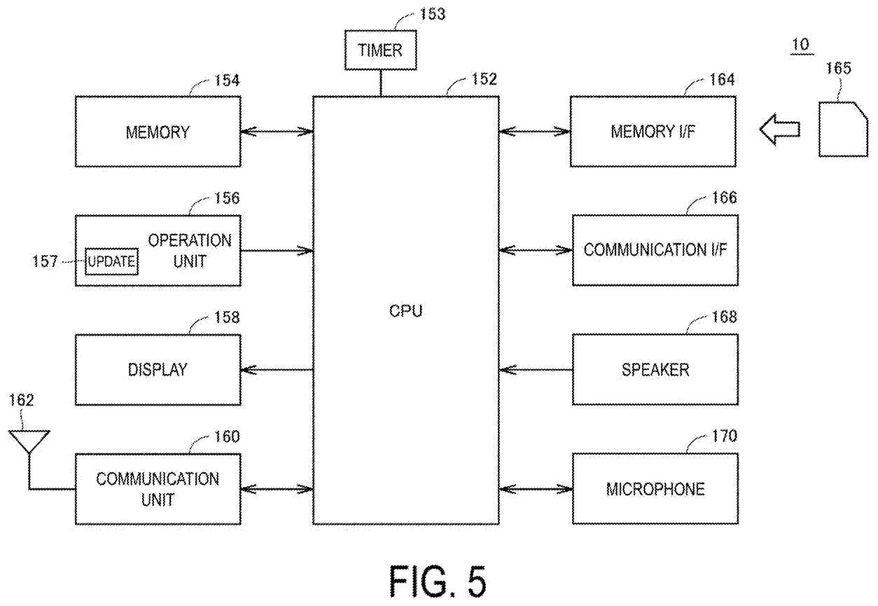

FIG. 5 is a configuration diagram of the terminal device 10 according to the first embodiment. With reference to FIG. 5, the terminal device 10 includes, as primary constituent elements, a CPU 152, a timer 153, a memory 154, an operation unit 156 that receives a user's operation to the terminal device 10, a display 158, a communication unit 160 for wireless communication via an antenna 162, a memory interface (I/F) 164, a communication interface (I/F) 166, a speaker 168 for audio output, and a microphone 170 for voice input.

The CPU 152 controls each component by executing programs stored in the memory 154.

A storage region of the memory 154 is constituted by random access memory (RAM), read-only memory (ROM), flash memory, a hard disk device, or the like. Programs to be executed by the CPU 152, data to be used by the CPU 152, and the like are stored in the memory 154.

The operation unit 156 receives an operation input to the terminal device 10. Typically, the operation unit 156 is configured to include a touch panel. The touch panel is provided on the display 158. The operation unit 156 may include a switch button and the like.

The operation unit 156 includes a button 157 that is operated by a user to instruct an update (change) of the treatment program of the low-frequency treatment device 20. The button 157 includes a hardware button or a software button displayed on the touch panel. When the button 157 is operated, the CPU 152 transmits a download request to the server 30. The download request includes a command to the server 30 requesting a parameter of a recommended treatment waveform described below. Details of the parameter of the recommended treatment waveform will be described later.

The communication unit 160 connects to a mobile communication network via the antenna 162, and transmits and receives signals for wireless communication. Accordingly, the terminal device 10 can communicate with other communication devices (for example, server 30 and other terminal device 10) via a mobile communication network such as Long Term Evolution (LTE), for example.

The memory interface 164 reads data from an external storage medium 165. The CPU 152 reads the data stored in the storage medium 165 via the memory interface 164 and stores the data in the memory 154. The CPU 152 reads the data from the memory 154 and stores the data in the external storage medium 165 via the memory interface 164.

The storage medium 165 may include a compact disc (CD), digital versatile disk (DVD), Blu-ray (trade name) disc (BD), universal serial bus (USB) memory, secure digital (SD) memory card, and the like that store programs or data in a non-volatile manner.

The communication interface (I/F) 166 is realized by using an adapter, connector, or the like to control communication between the terminal device 10 and the mobile device 21 or the low-frequency treatment device 20. In the present embodiment, Bluetooth (trade name) low energy (BLE) is employed as the communication method. However, the communication method may be a wireless communication method such as wireless LAN or a wired communication method using universal serial bus (USB) or the like.

### Configuration of Server 30

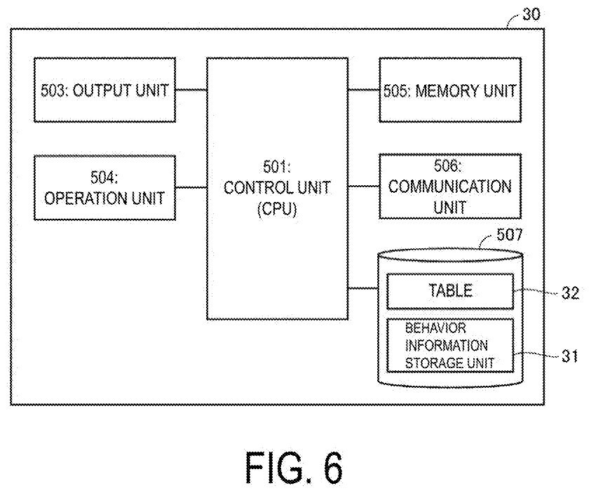

FIG. 6 is a configuration diagram of the server 30 according to the first embodiment. With reference to FIG. 6, the server 30 includes a control unit 501 including a CPU, an output unit 503 for outputting information, and an operation unit 504 including a button switch for receiving a user operation to the server 30. The server 30 further includes a communication unit 506 for communicating with the terminal device 10 and the like via the network 41, a memory unit 505 including ROM, RAM, or the like that stores programs and data, and a hard disc drive (HDD) 507.

The HDD 507 includes the behavior information storage unit 31 and the table 32. The behavior information storage unit 31 is a region for accumulating and storing behavior information received from the terminal device 10. The behavior information includes behavior information 60 based on the user's daily actions (hereinafter referred to as action-behavior information), and behavior information 61 based on user operation of the low-frequency treatment device 20 (hereinafter referred to as operation-behavior information). In the table 32, the information of the treatment waveform (more specifically, the parameters described above) is registered. Details of the behavior information storage unit 31 and the table 32 are described below.

Detection of User Behavior information (Action)

In order to obtain the operation-behavior information 61, the control unit 101 of the mobile device 21 determines the action of the user wearing the mobile device 21.

Specifically, in the first embodiment, with the mobile device 21 being a watch-type mobile device, the control unit 101 detects walking with arm swing based on an acceleration component from the acceleration sensor 110. Note that the type of action detected from the acceleration component is not limited to walking and may be, for example, running, the action of rotating an arm, or the action of rotating a shoulder.

Additionally, the control unit 101 detects whether the user is moving in an upright position in a train by combining the acceleration component from the acceleration sensor 110 and the surrounding sound collected by the microphone 112.

Additionally, the control unit 101 detects whether the user is climbing an mountain by combining the acceleration component from the acceleration sensor 110 and the altitude detected by the GPS unit 109.

The control unit 101 measures the amount of time spent performing the determined type of action (walking, moving while standing in a train, mountain climbing, and the like). The control unit 101 generates action-behavior information 60 including the type of action and the measured time spent, and transmits the action-behavior information 60 to the server 30 via the terminal device 10.

Note that in a case that the terminal device 10 is carried by the user, the terminal device 10 may detect the type of action and the time spent described above, and transmit the action-behavior information 60 including the detected type of action and the measured time spent to the server 30.

### Detection of User Behavior Information (Operation)

In the first embodiment, the user operates the low-frequency treatment device 20 via the operation unit 156 of the terminal device 10. The terminal device 10 transmits, to the low-frequency treatment device 20, control information, for example, to perform a specified treatment mode (treatment content) on the basis of the user operation content received via the operation unit 156. The treatment mode is specified, for example, by a combination of the treatment area, an electrical stimulation intensity, and a treatment time.

Based on a type of treatment mode included in the control information from the terminal device 10, the low-frequency treatment device 20 reads out a treatment mode program corresponding to the type from the storage unit, and execute the treatment mode program. In this way, components of the low-frequency treatment device 20 are controlled to perform the treatment content in accordance with the treatment mode specified by the user operation.

The terminal device 10 generates and transmits the operation-behavior information 61 to the server 30 when the treatment mode indicated by the user operation is performed. This operation-behavior information 61 includes the type of treatment mode specified by the user operation and the number of times that the treatment mode is performed (or the amount of time the treatment mode is performed).

### Functional Configuration of Server 30

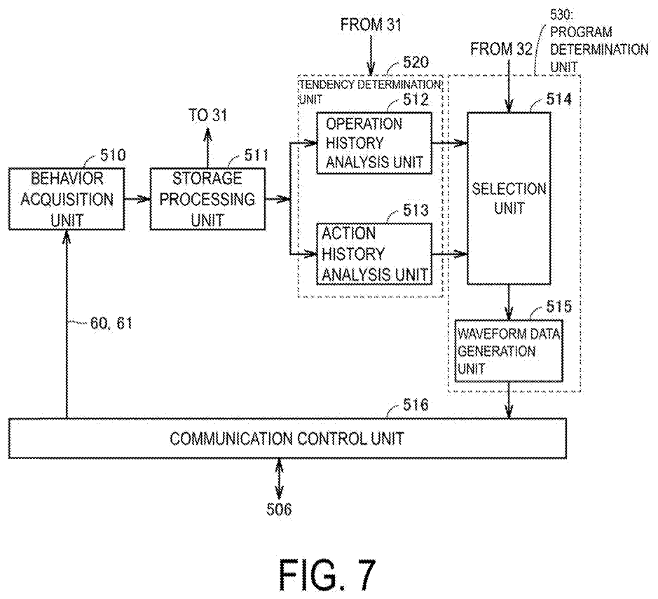

FIG. 7 is a diagram illustrating a functional configuration of the server 30 according to the first embodiment. The configuration of FIG. 7 illustrates primarily the function of determining the behavior tendency of the user from the behavior information (the action-behavior information 60 or the operation-behavior information 61) and determining a treatment program (more specifically, the parameters described above) that causes the low-frequency treatment device to perform a treatment content corresponding to the determined behavior tendency. The functions of each component are mainly realized by the control unit 501 executing a computer program. Note that the components are not limited to those realized by a computer program, and may be realized by a combination of a computer program and a circuit.

With reference to FIG. 7, the control unit 501 includes a communication control unit 516 for controlling the communication unit 506; a behavior acquisition unit 510 that outputs behavior information received via the communication control unit 516 to a storage processing unit 511 according to the order of the reception, the storage processing unit 511 that stores the behavior information output from the behavior acquisition unit 510 in the behavior information storage unit 31 according to the order of the reception, a tendency determination unit 520, and a program determination unit 530.

The tendency determination unit 520 includes an operation history analysis unit 512 and an action history analysis unit 513. The operation history analysis unit 512 analyzes the operation-behavior information 61 in the behavior information storage unit 31 and determines an operation tendency of the user of the low-frequency treatment device 20 on the basis of the results of the analysis. The operation tendency, to be explained in more detail, indicates the tendencies relating to selection of the treatment modes described above.

The action history analysis unit 513 analyzes the action-behavior information 60 in the behavior information storage unit 31 and determines a routine action tendency of the user on the basis of the results of the analysis.

The program determination unit 530 determines a recommended treatment program corresponding to the behavior tendency determined by the tendency determination unit 520. Specifically, the program determination unit 530 includes a selection unit 514 that searches the table 32 on the basis of the determined behavior tendency and selects a parameter of a recommended treatment waveform on the basis of the results of the search, and a waveform data generation unit 515. The waveform data generation unit 515 generates recommended waveform data including the selected parameter of a recommended waveform and outputs the generated recommended waveform data to the communication control unit 516. The communication control unit 516 controls the communication unit 506 to transmit the recommended waveform data to the terminal device 10.

### Description of Behavior Information Storage Unit 31

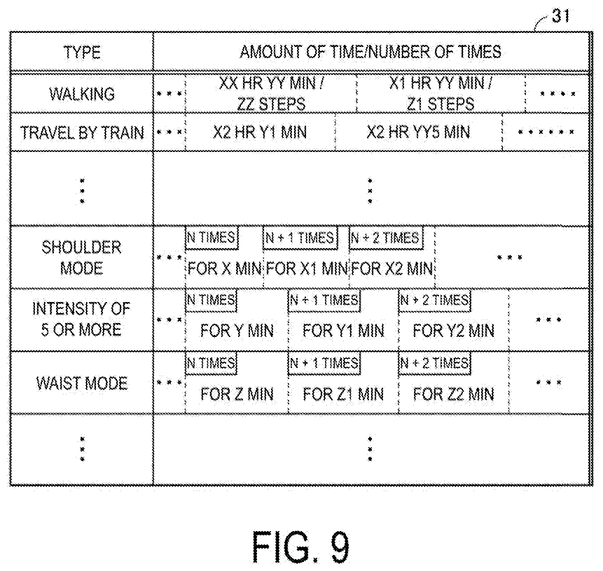

FIG. 9 is a diagram illustrating an example of the contents of the behavior information storage unit 31 according to the first embodiment. Referring to FIG. 9, the behavior information storage unit 31 contains a plurality of behavior types and an amount of time or the number of times the behavior is performed, associated with each type of behavior. If the type of behavior is a user action (for example, walking, moving while standing in a train, and the like), the time spent performing the action is associated. Also, whether the type of behavior is a user operation on the low-frequency treatment device 20 (specifying the shoulder mode, specifying a stimulation intensity of five or more, specifying the waist mode, and the like), the number of times (or amount of time) that the operation has been performed is associated.

Each time the storage processing unit 511 receives behavior information from the behavior acquisition unit 510, the storage processing unit 511 associates the type of behavior of the received behavior information with the amount of time or the number of times, and stores the behavior information in the behavior information storage unit 31. The storage processing unit 511 stores the behavior information according to the order of the reception, that is, in accordance with a chronological order of the actions and operations performed by the user. This allows the behavior information in the behavior information storage unit 31 to represent the history of user behavior.

### Description of Table 32

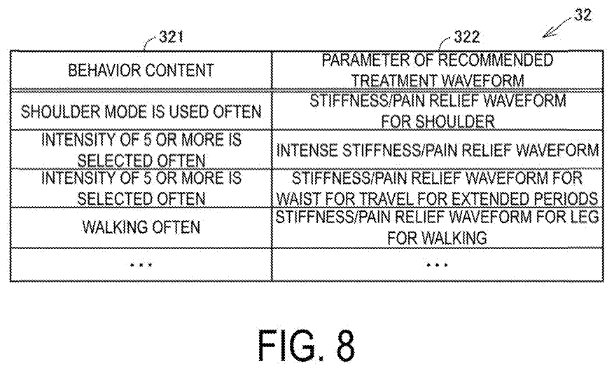

FIG. 8 is a diagram illustrating an example of the contents of the table 32 according to the first embodiment. Referring to FIG. 8, the table 32 includes behavior content 321 that indicates content of multiple types of behavior and a parameter 322 of a recommended treatment waveform associated with each behavior content 321. The information in the table 32 is acquired by experimentation or the like. Note that the parameter 322 may include the waveform parameters, voltage parameters, and time parameters described above.

### Flowchart of Processing

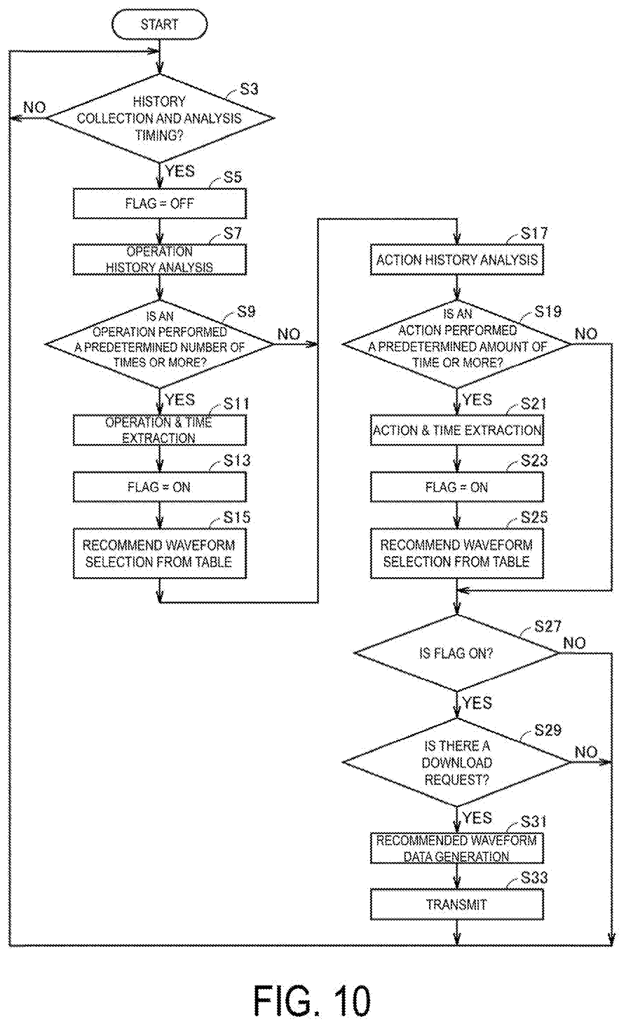

FIG. 10 is a flowchart of processing according to the first embodiment. The program according to this flowchart is stored in a storage unit of the server 30. The control unit 501 reads out the program from the storage unit, and executes the program that is read out.

Note that in the behavior information storage unit 31, action-behavior information 60 or operation-behavior information 61 received from the terminal device 10 is stored by the storage processing unit 511. Also, in the flowchart of processing in FIG. 10, a flag is used to indicate that a parameter of a recommended treatment waveform has been determined.

Referring to FIG. 10, the control unit 501 collects the behavior information in the behavior information storage unit 31 and determines whether it is time to make an analysis (step S3). In the first embodiment, for example, every day at 00:00 is set as the time for collection and analysis.

Every day, when the time is not 00:00 (NO in step S3), the control unit 501 repeats step S3, but sets the flag to ‘OFF’ (Step S5) when the time is 00:00 (YES in step S3).

The control unit 501 activates the operation history analysis unit 512 (step S7). The operation history analysis unit 512 analyzes the information in the behavior information storage unit 31 and determines, on the basis of the analysis results, whether there is a treatment mode, among the treatment modes of the behavior content 321, that has been operated (or performed) a predetermined number of times or more during a certain time period dating back from the present time (step S9). When the operation history analysis unit 512 determines that there are no treatment modes that have been operated the predetermined number of times or more (NO in step S9), the process proceeds to step S17.

On the other hand, when the operation history analysis unit 512 determines that there is a treatment mode that has been operated the predetermined number of times or more (YES in step S9), the operation history analysis unit 512, for each treatment mode determined in step S9, performs a process of extracting the number of times or the amount of time the operation is performed, and one treatment mode is determined on the basis of the results of the extraction process (step S11).

For example, if two or more treatment modes are determined in step S9, in step S11, the operation history analysis unit 512, for each treatment mode determined, extracts the amount of time the treatment mode is performed and determines one of the two or more treatment modes on the basis of the extracted amount of time. For example, the treatment mode with a greater value of the total length of time during which the treatment mode is performed is determined. In this way, a tendency of the user's operation for specifying the treatment modes during a certain time period dating backwards is determined.

The operation history analysis unit 512 sets the flag to ‘ON’ when the treatment mode is determined according to the tendency (step S13). The selection unit 514 searches the table 32 on the basis of the treatment mode determined in step S11 and selects the parameter 322 of the recommended treatment waveform associated with the behavior content 321 that matches the treatment mode on the basis of the results of the search (step S15). The selected parameter 322 of the recommended treatment waveform is output to the waveform data generation unit 515. Thereafter, the process proceeds to step S17.

In step S17, the control unit 501 activates the action history analysis unit 513 (step S17). The action history analysis unit 513 analyzes the information in the behavior information storage unit 31 and determines, on the basis of the analysis results, whether there is an action, among the types of actions in the behavior content 321, that is performed for a predetermined amount of time or more during a certain time period dating back from the present time (step S19). When the action history analysis unit 513 determines that there are no actions that have been performed for the predetermined amount of time or more (NO in step S19), the process proceeds to step S27.

On the other hand, when the action history analysis unit 513 determines that there is an action that has been performed for the predetermined amount of time or more (YES in step S19), the action history analysis unit 513, for each action determined in step S19, performs a process of extracting the amount of time the action is performed from the behavior information storage unit 31, and one type of action is determined on the basis of the results of the extraction process (step S21).

For example, if two or more types of actions are determined in step S19, in step S21, the action history analysis unit 513, for each type of action determined, extracts the amount of time the action is performed from the behavior information storage unit 31 and determines one of the two or more types of actions on the basis of the extracted amount of time. For example, the action with a greater value of the total length of time during which the action is performed is determined. In this way, a tendency of a user's action during a certain time period dating backwards is determined.

The action history analysis unit 513 sets the flag to ‘ON’ when the type of action is determined according to the tendency (step S23). The selection unit 514 searches the table 32 on the basis of the type of action determined in step S21 and selects the parameter 322 of the recommended treatment waveform associated with the behavior content 321 that matches the type of action on the basis of the results of the search (step S25). The selected parameter 322 of the recommended treatment waveform is output to the waveform data generation unit 515. Thereafter, the process proceeds to step S27.

In step S27, the control unit 501 determines whether or not the flag is ‘ON’ (step S27). By determining whether or not the flag is ‘ON’, in step S15 or step S25, whether or not the parameter 322 of the recommended treatment waveform has been selected is determined.

If it is determined that the flag is not ‘ON’ (NO in step S27), the control unit 501 returns to step S3 and performs the subsequent processing in a similar manner. On the other hand, if the flag is determined to be ‘OFF’ (YES in step S27), the control unit 501 determines whether or not a download request for the parameter 322 of the recommended treatment waveform has been received from the terminal device 10 (step S29).

If it is determined that the download request has not been received (NO in step S29), the control unit 501 returns to step S3 and performs the subsequent processing. On the other hand, if the control unit 501 determines that a download request has been received (YES in step S29), the waveform data generation unit 515 generates a recommended waveform data including the parameter 322 of the recommended treatment waveform and outputs the generated recommended waveform data to the communication control unit 516 (step S31).

The communication control unit 516 transmits the recommended waveform data from the waveform data generation unit 515 to the terminal device 10 (step S33).

On the basis of the recommended waveform data from the server 30, the terminal device 10 generates control information including the parameter 322 of the recommended treatment waveform and a command for setting the parameter 322 in the treatment program, and transmits the control information to the low-frequency treatment device 20. In the low-frequency treatment device 20, the treatment program according to the control information from the terminal device 10 is executed to perform a treatment content in accordance with the behavior tendency of the user.

### MODIFIED EXAMPLE 1

A modified example of the first embodiment described above will be described. In the first embodiment, the server 30 determines the behavior tendency on the basis of the behavior information in the behavior information storage unit 31, and the parameter 322 of the recommended treatment waveform is determined on the basis of the determined tendencies. However, in Modified Example 1, the terminal device 10 or the low-frequency treatment device 20 may perform such processing.

For example, the server 30 may determine the behavior tendency, and the terminal device 10 or the low-frequency treatment device 20 may determine the parameter 322 of the recommended treatment waveform on the basis of the behavior tendency received from the server 30.

Additionally, for example, the terminal device 10 may determine the behavior tendency from the behavior information of the user, and the low-frequency treatment device 20 may determine the parameter 322 of the recommended treatment waveform on the basis of the behavior tendency received from the terminal device 10.

Additionally, for example, the terminal device 10 may determine the behavior tendency on the basis of the behavior information, determine the parameter 322 of the recommended treatment waveform on the basis of the determined behavior tendency, and transmit the recommended waveform data including the determined parameter 322 to the low-frequency treatment device 20.

For example, the low-frequency treatment device 20 collects the behavior information of the user and stores the behavior information in the storage unit 206. The control unit 203 may determine the behavior tendency on the basis of the behavior information in the storage unit 206 and determine the parameter 322 of the recommended treatment waveform from the table of the storage unit 206 on the basis of the determined tendencies. In this case, the stand-alone low-frequency treatment device 20 can operate independent of the terminal device 10 and the server 30, thus causing the low-frequency treatment device 20 to perform a treatment program according to the behavior tendency of the user.

### MODIFIED EXAMPLE 2

A modified example of the first embodiment described above will be described. In Modified Example 2, when the server 30 determines the behavior tendency of the user, the server 30 queries the low-frequency treatment device 20 as to whether or not the parameter 322 of the recommended treatment waveform corresponding to the determined behavior tendency is included. The server 30 transmits the query to the low-frequency treatment device 20 via the terminal device 10, for example.

The server 30 performs a similar query to the terminal device 10 when the server 30 receives a response from the low-frequency treatment device 20 that “the corresponding parameter 322 is not included”. The server 30 controls the terminal device 10 to transmit the recommended waveform data including the corresponding parameter 322 to the low-frequency treatment device 20 when a response of “the corresponding parameter 322 is included” is received from the terminal device 10.

Note that in a case that the server 30 performs a query about the parameter 322 corresponding to the behavior tendency of the user and the low-frequency treatment device 20 includes the parameter 322, the low-frequency treatment device 20 executes the treatment program with the parameter 322 set therein to perform the treatment content according to the behavior tendency of the user.

### MODIFIED EXAMPLE 3

In the first embodiment, the treatment program is determined (changed) by setting the parameter 322 of the recommended treatment waveform in the treatment program (changing the original parameter). However, the method of determining (changing) the treatment program is not limited to a method of changing the parameter, and the treatment program may be selectively changed, for example.

A case of selectively changing the treatment program will be described. In the storage unit 206 of the low-frequency treatment device 20, a treatment program is stored corresponding to each set of parameters of the recommended waveform. When control information (including parameters) is received from the terminal device 10, the low-frequency treatment device 20 searches the storage unit 206 on the basis of the parameters of the control information. On the basis of the results of the search, the low-frequency treatment device 20 reads out, from the storage unit, a treatment program corresponding to the parameter, executes the treatment program that is read out, and outputs a PWM control signal.

### Second Embodiment

The method of determining the parameter of the recommended treatment waveform described by using the flowchart of FIG. 10 described above may be provided as a program that a computer (CPU) executes. The program for realizing this method is stored in the server 30 or the terminal device 10 or a storage unit of the low-frequency treatment device 20. The program may be downloaded via a communication line to the server 30 or the terminal device 10 or the storage unit of the low-frequency treatment device 20.

Alternatively, in the terminal device 10, for example, a program that is read out from the external storage medium 165 may be loaded (stored) into the memory 154 via the memory I/F 164. In addition, in the server 30 or the low-frequency treatment device 20, a program download using such an external storage medium may be performed.

The embodiments described herein are illustrative in all respects and are not intended as limitations. The scope of the present invention is indicated not by the descriptions above but by the claims and includes all meaning equivalent to the scope and changes within the scope.

### REFERENCE SIGNS LIST

1 Information processing system
2 Pad
2Y Treatment portion
2a Conductive layer
3 Holder
4 Main body portion
4a Case
5 Engagement portion
10 Terminal device
20 Low-frequency treatment device
21 Mobile device
30 Server
31 Behavior information storage unit
32 Table
41, 43 Network
48S Switch
60, 61 Behavior information
101, 203, 501 Control unit
110 Acceleration sensor
111 Blood pressure measurement unit
112, 170 Microphone
157 Button
165 Storage medium
204 Current generation unit
321 Behavior content
322 Parameter
510 Behavior acquisition unit
511 Storage processing unit
512 Operation history analysis unit
513 Action history analysis unit
514 Selection unit
515 Waveform data generation unit
516 Communication control unit
520 Tendency determination unit
530 Program determination unit

1. An information processing device, comprising:
a control unit configured to control the information processing device, wherein
the control unit comprises
a storage processing unit configured to acquire behavior information indicating a content of a behavior of a user of an electrical treatment device and store the behavior information in a storage unit, the behavior of the user being an action of the user itself or an operation by the user to the electrical treatment device, and
the control unit further comprises
a tendency determination unit configured to determine a behavior tendency of the user, which is a tendency relating to an action of the user itself or a tendency relating to an operation by the user to the electrical treatment device, on the basis of the behavior information indicating the content of the behavior of the user stored in the storage unit, and
a program determination unit configured to determine a treatment program for causing the electrical treatment device to perform a treatment content corresponding to the behavior tendency.

2. The information processing device according to claim 1, wherein
the electrical treatment device comprises a current generation unit configured to generate a current to be output to an area of a body according to the treatment program via an electrode portion configured to come into contact with the area, and
the treatment program comprises a parameter for specifying a current waveform.

3. The information processing device according to claim 1, wherein
in a case that the behavior is an action of the user itself,
the behavior information comprises a type of the action of the user and an amount of time or the number of times the action is performed.

4. The information processing device according to claim 1, wherein
in a case that the behavior is a user operation to the electrical treatment device,
the behavior information comprises a type of treatment mode to be specified by the user operation and an amount of time or the number of times the treatment mode has been specified.

5. An electrical treatment device, comprising:
a control unit configured to control the electrical treatment device, wherein
the control unit is configured to acquire information indicating a behavior tendency of a user, which is a tendency relating to an action of the user itself of the electrical treatment device or a tendency relating to an operation by the user to the electrical treatment device, and determine a treatment program for causing the electrical treatment device to perform a treatment content corresponding to the behavior tendency of the user indicated by the information acquired.

6. The electrical treatment device according to claim 5, wherein
the electrical treatment device comprises a current generation unit configured to generate a current to be output to an area of a body according to the treatment program via an electrode portion configured to come into contact with the area, and
the treatment program comprises a parameter for specifying a current waveform.

7. A system, comprising:
an information processing device; and
an electrical treatment device that communicates with the information processing device, wherein
the information processing device comprises a control unit configured to control the information processing device,
the control unit comprises
a tendency determination unit configured to determine a behavior tendency of a user on the basis of behavior information indicating a content of a behavior of a user stored in a storage unit, the behavior of the user being an action of the user itself or an operation by the user to the electrical treatment device, and
the control unit further comprises
a program determination unit configured to determine a treatment program for causing the electrical treatment device to perform a treatment content corresponding to the behavior tendency of the user, which is a tendency relating to an action of the user itself or a tendency relating to an operation by the user to the electrical treatment device, and
a transmission unit configured to transmit information of the treatment program determined to the electrical treatment device.

8. The system according to claim 7, wherein
the electrical treatment device comprises a current generation unit configured to generate a current to be output to an area of a body according to the treatment program via an electrode portion configured to come into contact with the area, and
the treatment program comprises a parameter for specifying a current waveform.

9. A program for causing a computer to execute an information processing method, wherein
the information processing method comprises
acquiring behavior information indicating a content of a behavior of a user of an electrical treatment device and storing the behavior information in a storage unit, the behavior of the user being an action of the user itself or an operation by the user to the electrical treatment device, and
the information processing method further comprises
determining a behavior tendency of the user, which is a tendency relating to an action of the user itself or a tendency relating to an operation by the user to the electrical treatment device, on the basis of the behavior information stored in the storage unit, and
determining a treatment program for causing the electrical treatment device to perform a treatment content corresponding to the behavior tendency.

10. The program according to claim 9, wherein
the electrical treatment device comprises a current generation unit configured to generate a current to be output to an area of a body according to the treatment program via an electrode portion configured to come into contact with the area, and
the treatment program comprises a parameter for specifying a current waveform.

11. The information processing device according to claim 2, wherein
in a case that the behavior is an action of the user itself,
the behavior information comprises a type of the action of the user and an amount of time or the number of times the action is performed.

12. The information processing device according to claim 2, wherein
in a case that the behavior is a user operation to the electrical treatment device,
the behavior information comprises a type of treatment mode to be specified by the user operation and an amount of time or the number of times the treatment mode has been specified.

13. The information processing device according to claim 3, wherein
in a case that the behavior is a user operation to the electrical treatment device,
the behavior information comprises a type of treatment mode to be specified by the user operation and an amount of time or the number of times the treatment mode has been specified.

14. The information processing device according to claim 11, wherein
in a case that the behavior is a user operation to the electrical treatment device,
the behavior information comprises a type of treatment mode to be specified by the user operation and an amount of time or the number of times the treatment mode has been specified.
